(19) United States
(12) Patent Application Publication
(10) Pub. No.: US 2026/0020922 A1
(43) Pub. Date: Jan. 22, 2026

(54) SYSTEMS AND METHODS OF MOVING A MEDICAL TOOL WITH A TARGET IN A VISUALIZATION OR ROBOTIC SYSTEM FOR HIGHER YIELDS

(71) Applicant: COVIDIEN LP, Mansfield, MA (US)

(72) Inventor: William J. DICKHANS, Longmont, CO (US)

(21) Appl. No.: 19/116,554

(22) PCT Filed: Oct. 5, 2023
(86) PCT No.: PCT/IB2023/060018

§ 371 Date: Mar. 28, 2025

Related U.S. Application Data
(60) Provisional application No. 63/416,338, filed on Oct. 14, 2022.

(51) Int. Cl. A61B 34/30; A61B 5/00; A61B 5/08; A61B 5/113; A61B 34/20; A61B 90/00

(52) U.S. Cl. CPC A61B 34/30; A61B 5/0077; A61B 5/0816; A61B 5/1135; A61B 5/742; A61B 34/20; A61B 90/37; A61B 2034/2051; A61B 2034/305; A61B 2090/374; A61B 2090/3762

(57) ABSTRACT

Visualization and robotic systems and methods utilize preoperative three dimensional (3D) images of patient motion and intraoperative, real-time patient motion information to show a target moving relative to medical tool or to control a robotic medical tool in real-time to track the target while the target is biopsied or treated. The systems and methods involve receiving preoperative 3D images of patient motion, displaying guidance for or controlling a robotic tool for navigating a medical tool near the target based on information from a position sensor disposed on the medical tool, tracking intraoperative 3D patient motion using motion sensors disposed on the patient, determining 3D target motion based on the preoperative 3D images and the tracked patient motion, and controlling the medical tool with the robotic tool to track the 3D target motion or displaying the 3D target motion relative to the medical tool.

### FIELD

This disclosure relates to the field of visualization and navigation of medical tools, such as biopsy or ablation tools, relative to targets, and tracking real-time motion of the targets during navigation or treatment of the targets with medical tools.

### BACKGROUND

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and to a target for biopsy or treatment. In some procedures, preoperative scans may be utilized for target identification and intraoperative guidance. In some cases, real-time imaging or intraoperative imaging may also be required to obtain a more accurate and current image of the target area and an endoluminal medical tool used to biopsy or treat tissue in the target area. Furthermore, real-time image data displaying the current location of a medical device with respect to the target and its surroundings may be needed to navigate the medical device to the target in a safe and accurate manner (e.g., without causing damage to other organs or tissue). Real-time, intraoperative imaging, however, may expose the patient to unnecessary and/or potentially unhealthy amounts of X-ray radiation.

An endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use previously acquired MRI data or CT image data to generate a three-dimensional (3D) rendering, model, or volume of the particular body part such as the lungs.

The resulting volume generated from the MRI scan or CT scan is then utilized to create a navigation plan to facilitate the advancement of a navigation catheter (or other suitable medical tool) through a bronchoscope and a branch of the bronchus of a patient to an area of interest. A locating or tracking system, such as an electromagnetic (EM) tracking system, may be utilized in conjunction with, for example, CT data, to facilitate guidance of the navigation catheter through branches of the bronchus to the area of interest. In certain instances, the navigation catheter may be positioned within one of the airways of the branched luminal networks adjacent to, or within, the area of interest to provide access for one or more medical tools.

However, a 3D volume of a patient's lungs, generated from previously acquired scans, such as CT scans, may not provide a basis sufficient for accurate guiding of medical devices or tools to a target during a navigation procedure. In some cases, the inaccuracy is caused by deformation of the patient's lungs during the procedure relative to the lungs at the time of the acquisition of the previously acquired CT data. This deformation (CT-to-Body divergence) may be caused by many different factors including, for example, changes in the body when transitioning from between a sedated state and a non-sedated state, the bronchoscope changing the patient's pose, the bronchoscope pushing the tissue, different lung volumes (e.g., the CT scans are acquired during inhale while navigation is performed during breathing), different beds, different days, etc. This deformation may lead to significant motion of a target, making it challenging to align a medical tool with the target in order to safely and accurately biopsy or treat tissue of the target.

Thus, systems and methods are needed to account for motion of a patient during an in-vivo navigation and biopsy or treatment procedures. Furthermore, to navigate a medical tool safely and accurately to a remote target and biopsy or treat the remote target with the medical tool, either by a surgical robotic system or by a clinician using a guidance system, the systems should track the target during the motion of the patient's body, e.g., motion of the patient's chest during respiration, while minimizing the patient's exposure to intraoperative X-ray radiation.

### SUMMARY

The techniques of this disclosure generally relate to systems and methods showing a target moving relative to medical tool or controlling a robotic medical tool in real-time to track the target while the target is biopsied or treated using preoperative three dimensional (3D) images of patient motion and intraoperative, real-time patient motion information.

In one aspect, the disclosure provides a method of controlling a medical tool to track the 3D motion of a target in a patient. The method includes receiving preoperative three dimensional (3D) images of motion of a patient including a target. The method also includes navigating a medical tool near the target based on information from a position sensor disposed on the medical tool, receiving patient motion information, tracking intraoperative 3D motion of the patient based on the patient motion information yielding tracked patient motion, and determining 3D motion of the target in the patient based on the preoperative 3D images and the tracked patient motion. The method also includes controlling the medical tool to track the 3D motion of the target.

Implementations of the method may also include one or more of the following features. The preoperative 3D images may be computed tomography (CT) images, cone beam computed tomography (CBCT) images, or magnetic resonance imaging (MRI) images. The patient motion information may be received from electromagnetic (EM) motion sensors or an anesthesia machine. The preoperative 3D images may be captured using functional respiratory imaging (FRI). Determining the 3D motion of the target in the patient may include registering the preoperative 3D images with the tracked patient motion.

In another aspect, the disclosure provides an endoluminal navigation method. The endoluminal navigation method includes receiving preoperative 3D images of motion of a patient including a target and displaying guidance for navigating a medical tool near the target based on the preoperative 3D images and information from a position sensor disposed on the medical tool. The endoluminal navigation method also includes receiving patient motion information and tracking motion of the patient based on the patient motion information, yielding tracked patient motion. The endoluminal navigation method also includes determining 3D motion of the target in the patient based on the preoperative 3D images and the tracked patient motion, and displaying the 3D motion of the target relative to a tip of the medical tool.

Implementations of the endoluminal navigation method may also include one or more of the following features. The endoluminal navigation method may include capturing the preoperative 3D images during a respiratory cycle of the patient. The endoluminal navigation method may include displaying an indicator of at least one direction in which to navigate the medical tool to reach the target. The endoluminal navigation method may include segmenting the target from the preoperative 3D images yielding segmented targets and determining positions of the target in a reference frame of the preoperative 3D images based on the segmented targets. The endoluminal navigation method may include registering the preoperative 3D images to tracked patient motion.

In still another aspect, the disclosure provides a robotic endoluminal navigation system. The robotic endoluminal navigation system includes a robotic arm that holds and navigates a medical tool, an electromagnetic (EM) field generator that generates an electromagnetic field, a first EM sensor disposed at a tip of the medical tool, and one or more second EM sensors disposed on a patient. The robotic endoluminal navigation system also includes a processor and a memory having stored thereon instructions, which, when executed by the processor, cause the processor to receive preoperative 3D images of motion of the patient and track navigation of the medical tool towards a target using the first EM sensor. The instructions, when executed by the processor, may also cause the processor to intraoperatively track motion of the patient using the one or more second EM sensors disposed on the patient yielding tracked patient motion. The instructions, when executed by the processor, may also cause the processor to determine 3D motion of the target in the patient based on the preoperative 3D images and the tracked patient motion, and control the medical tool to align with the target during motion of the patient using the first EM sensor and the 3D motion of the target.

Implementations of the robotic endoluminal navigation system may also include one or more of the following features. The preoperative 3D images may be captured during at least one respiratory cycle of the patient. The instructions, when executed by the processor, may cause the processor to control the robotic arm to navigate the medical tool towards the target during patient motion. The one or more second EM sensors may be disposed on the patient's chest and may track the motion of the patient's chest during at least one respiratory cycle. The instructions, when executed by the processor, may cause the processor to control the robotic arm to navigate the medical tool through a luminal network of the patient. The medical tool may be an extended working channel or a biopsy tool.

In still another aspect, the disclosure provides an endoluminal navigation system. The endoluminal navigation system includes an electromagnetic (EM) field generator that generates an electromagnetic field, a first EM sensor disposed at a tip of a medical tool, one or more second EM sensors disposed on a patient's chest, and a display. The endoluminal navigation system also includes a processor, and a memory having stored thereon instructions, which, when executed by the processor, cause the processor to receive preoperative 3D images of motion of the patient, track navigation of the medical tool towards a target using the first EM sensor, intraoperatively track motion of the patient using the one or more second EM sensors disposed on the patient's chest yielding tracked patient motion, determine 3D motion of the target in the patient based on the preoperative 3D images and the tracked patient motion, and display on the display the target and the tip of the medical tool relative to the target during motion of the patient using the first EM sensor and the 3D motion of the target.

Implementations of the endoluminal navigation system may also include one or more of the following features. The instructions, when executed by the processor, may cause the processor to display an indicator of at least one direction in which to navigate the medical tool to reach the target. The instructions, when executed by the processor, may cause the processor to segment the target from the preoperative 3D images. The instructions, when executed by the processor, may cause the processor to register the preoperative 3D images to the 3D motion of the target.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects of the disclosure are described hereinbelow with references to the drawings, wherein:

FIG. 1 is a diagram of a system for navigating to targets via luminal networks in accordance with the disclosure;

FIG. 2 is a flowchart of an example of a method for visualizing a medical tool tracking a target during motion of the patient in accordance with the disclosure;

FIG. 3 is a screen shot of an example of a navigation user interface showing a medical tool tip tracking a moving target in accordance with the disclosure;

FIG. 4 is a block diagram that illustrates a robotic surgical system;

FIG. 5 is a system block diagram that illustrates a robotic surgical control system for controlling the robotic surgical system of FIG. 4;

FIG. 6 is a flowchart of an example of a method for controlling a robotic arm to orient and advance a catheter relative to a moving target during respiration of a patient in accordance with the disclosure; and

FIG. 7 is a diagram of a system for visualizing or controlling a medical tool relative to a target during real-time navigation and use of the medical tool and during motion of the patient in accordance with the disclosure.

### DETAILED DESCRIPTION

A clinician may use a fluoroscopic imaging system, for example, to visualize the intraoperative navigation of a medical tool, e.g., a biopsy tool, and confirm the placement of the medical tool after it has been navigated to a desired location, e.g., near a target. However, although fluoroscopic images show highly dense objects, such as metal tools, bones, and large soft-tissue objects, e.g., the heart, the fluoroscopic images may not clearly show small soft-tissue objects of interest, such as lesions. Furthermore, the fluoroscopic images are two-dimensional projections. Therefore, an X-ray volumetric reconstruction is needed to enable identification of soft tissue objects and navigation of medical tools to those objects.

Several solutions exist that provide volumetric reconstruction. One solution is a CT imaging system, which algorithmically combines multiple X-ray projections from known, calibrated X-ray source positions into a volume, in which soft tissues are more visible. For example, a CT imaging system can be used with iterative scans during a procedure to provide guidance through the body until the medical tool reaches the target. This is a tedious procedure, as it requires several full CT scans, a dedicated CT room, and blind navigation between scans. In addition, each scan requires the staff to leave the room due to high levels of ionizing radiation and exposes the patient to the radiation. Another solution is a cone-beam CT imaging system. However, the cone-beam CT machine is expensive and, like the CT imaging system, only provides blind navigation between scans, requires multiple iterations for navigation, and requires the staff to leave the room. In some cases, the benefits of CT imaging and fluoroscopic imaging may be combined, e.g., by using preoperative CT imaging and intraoperative fluoroscopic imaging, to help clinicians or surgical robots navigate medical tools to targets, including small soft-tissue objects. Ideally, however, use of the CT and fluoroscopic imaging should be minimized in order to minimize human exposure to X-ray radiation. Aspects of the disclosure are aimed at minimizing human exposure to X-ray radiation.

In an electromagnetic navigation procedure, planning, registration, and navigation, which may involve navigation of a locatable guide within an extended working channel, are performed to ensure that a medical tool, e.g., a biopsy tool, follows a planned path to reach a target, e.g., a lesion, so that a biopsy or treatment of the target can be performed. Following the navigation phase, fluoroscopic images may be captured and utilized in a local registration process to reduce CT-to-body divergence. After the local registration process, the locatable guide may be removed from the extended working channel and a medical device, e.g., a biopsy tool, is introduced into the extended working channel and navigated to the target to perform the biopsy or treatment of the target, e.g., the lesion.

In navigating the medical device to the target, clinicians may use a live 2D fluoroscopic view to visualize the position of the medical tool relative to the target. While the medical tool may be visible in the live fluoroscopic view, some targets, e.g., lesions, may not be visible in the live fluoroscopic view. Moreover, the user interfaces that are used to advance or navigate a medical tool towards the target do not provide enough information regarding the medical tool relative to the moving target, including when the medical tool is near or aligned with the target.

Biopsy yields are tracked as the metric for evaluating different biopsy systems and methods. For example, biopsy yields for computed tomography (CT) image-guided transthoracic needle aspirations (TTNAs) are 90% or higher. Endoluminal biopsies have hovered between about 75% and 90%. Endoluminal biopsies, however, provide at least the option of staging the patient, e.g., by sampling the lymph nodes. Recently, techniques, such as tool-in-lesion, target overlay, and CT-to-body divergence correction have been used to increase biopsy yields. However, the target remains static on the overlays during biopsy sampling even though the target is moving in the 3D space of the lung.

This disclosure features systems and methods that move a medical tool, e.g., the distal tip of a biopsy catheter, in real time with the target on a display of a user interface or via control of a robotic arm holding the medical tool to ensure the medical tool can safely and accurately biopsy or treat the target tissue. For example, the systems and methods of the disclosure move a biopsy catheter with the target to ensure that the biopsy catheter takes samples of the target and not of other tissue, which may lead to complications such as a pneumothorax. In the case of biopsy procedures, moving the distal tip of a biopsy catheter with the target may lead to higher biopsy yields.

These systems and methods involve capturing preprocedural or preoperative 3D images, e.g., preoperative CT images, during patient motion, e.g., during one or more respiratory cycles. For example, the system and methods may use aspects of functional respiratory imaging (FRI). This allows for 3D motion information of the target to be input to and used by the endoluminal navigation system to guide a clinician or a robotic arm to navigate and control the medical tool to safely and accurately biopsy or treat a target. The preprocedural data with FRI along with real time data from sensors on the patient and tracking of the respiratory cycle would allow for accurate understand of 3D target movement in the chest.

Once the endoluminal catheter is at the target position, tracking may enable either showing the target moving relative to the catheter, e.g., a static catheter, or controlling a robotic arm and/or end-effector to manipulate a catheter in real-time to track the target while biopsies are being taken or treatment is being performed. In the case of biopsy procedures, this would allow for samples of the target to be taken for every biopsy.

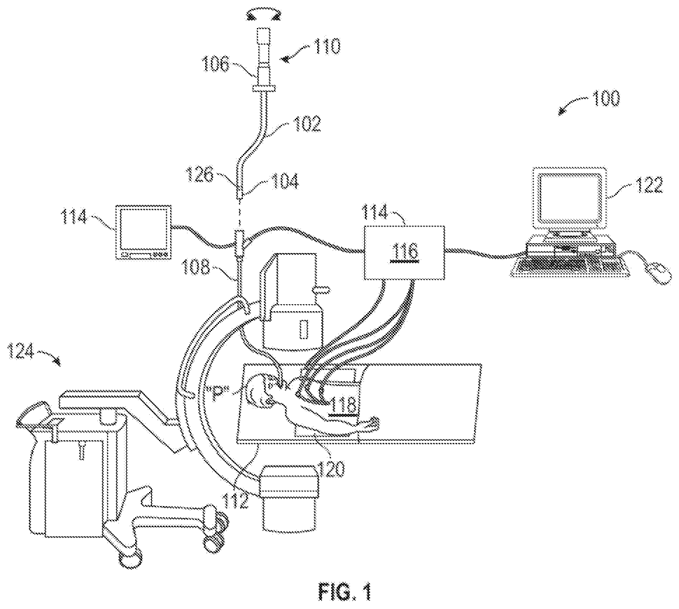

In accordance with aspects of the disclosure, the visualization and/or robotic control of intra-body navigation of a medical tool, e.g., a biopsy tool, towards a moving target, e.g., a lesion, may be a portion of a larger workflow of a navigation system, such as an electromagnetic navigation system. FIG. 1 is a perspective view of an example of a system for facilitating navigation of a medical tool, e.g., a biopsy tool, to a soft-tissue target via airways of the lungs. The system 100 may optionally be configured to generate a three-dimensional (3D) reconstruction of the target area from 2D fluoroscopic images. In the case where the system 100 is configured to generate a 3D reconstruction, intraoperative 2D fluoroscopic images may be captured only during critical parts of a procedure, e.g., to confirm placement of a medical tool in a target, in order to minimize human exposure to X-ray radiation. The system 100 may be further configured to facilitate approach of a medical tool to the target area by using Electromagnetic Navigation Bronchoscopy (ENB) and for determining the location of a medical tool with respect to the target.

One aspect of the system 100 is a software component for reviewing of computed tomography (CT) image data that has been acquired separately from system 100. The review of the CT image data allows a user to identify one or more targets, plan a pathway to an identified target (planning phase), navigate a catheter 102 to the target (navigation phase) using a user interface, and confirming placement of a sensor 104 relative to the target. One such EMN system is the ELECTROMAGNETIC NAVIGATION BRONCHOSCOPY® system, which is referred to as ENB, currently sold by Medtronic PLC. The target may be tissue of interest identified by review of the CT image data during the planning phase. Following navigation, a medical tool, such as a biopsy tool or other tool, may be inserted into the catheter 102 to obtain a tissue sample from the tissue located at, or proximate to, the target.

As shown in FIG. 1, the catheter 102 is part of a catheter guide assembly 106. In practice, the catheter 102 is inserted into a bronchoscope 108 for access to a luminal network of the patient P. Specifically, the catheter 102 of catheter guide assembly 106 may be inserted into a working channel of the bronchoscope 108 for navigation through a patient's luminal network. A locatable guide 110, including an electromagnetic (EM) sensor 104 is inserted into the catheter 102 and locked into position such that the sensor 104 extends a desired distance beyond the distal tip of the catheter 102. The position and orientation of the sensor 104 relative to the reference coordinate system, and thus the distal portion of the catheter 102, within an electromagnetic field can be derived. Catheter guide assemblies 106 are currently marketed and sold by Medtronic PLC under the brand names SUPERDIMENSION® Procedure Kits, or EDGE™ Procedure Kits, and are contemplated as useable with the disclosure.

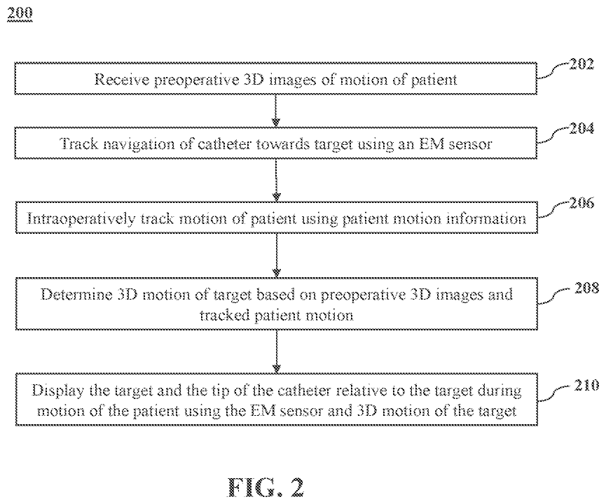

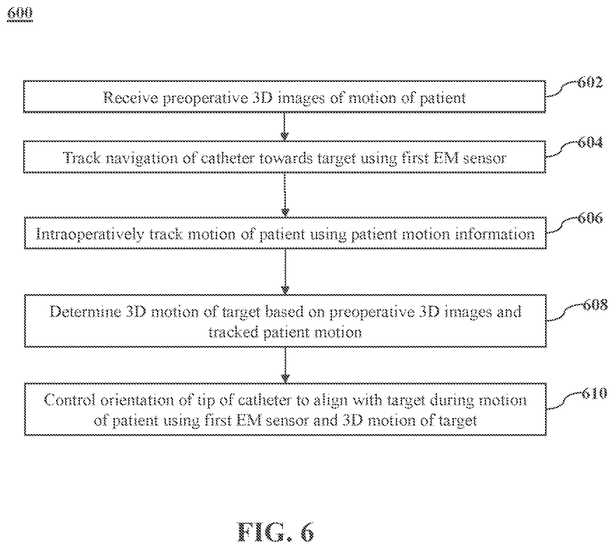

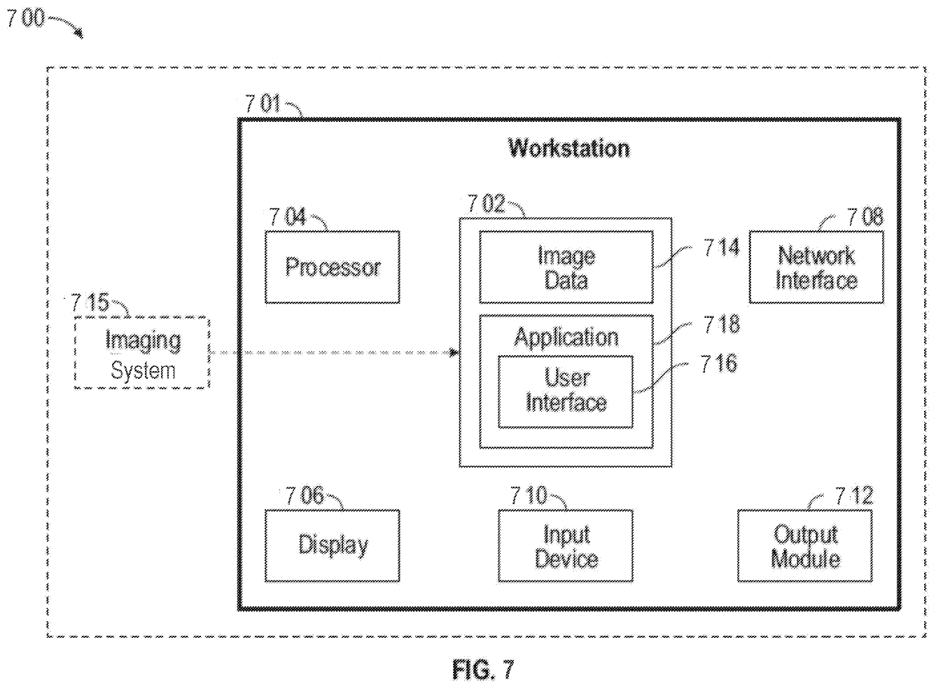

The system 100 generally includes an operating table 112 configured to support a patient P, a bronchoscope 108 configured for insertion through patient P's mouth into patient P's airways; monitoring equipment 114 coupled to the bronchoscope 108 (e.g., a video display, for displaying the video images received from the video imaging system of bronchoscope 108); a locating or tracking system 114 including a locating module 116, patient motion sensors 118, and a transmitter mat 120, which may include multiple markers; and a computer system 122 including software and/or hardware used to facilitate identification of a target, pathway planning to the target, navigation of a medical tool to the moving target, and/or confirmation and/or determination of placement of the catheter 102, or a suitable medical tool therethrough, relative to the target. The computer system 122 may be similar to workstation 701 of FIG. 7 and may be configured to execute the methods of the disclosure including the methods of FIGS. 2 and 6.

An imaging system 124 capable of acquiring fluoroscopic or x-ray images or video of the patient P is optionally included in some aspects of the system 100. The images, sequence of images, or video captured by the imaging system 124 may be stored within the imaging system 124 or transmitted to the computer system 122 for storage, processing, and display. Additionally, imaging system 124 may move relative to the patient P so that images may be acquired from different angles or perspectives relative to patient P to create a sequence of 2D images, such as a video. The pose of the imaging system 124 relative to the patient P and while capturing the images may be estimated via markers incorporated with the transmitter mat 120. The markers are positioned under patient P, between patient P and operating table 112 and between patient P and a radiation source or a sensing unit of the imaging system 124. The markers incorporated with the transmitter mat 120 may be two separate elements which may be coupled in a fixed manner or alternatively may be manufactured as a single unit. The imaging system 124 may include a single imaging system or more than one imaging systems. As illustrated in FIG. 1, the imaging system 124 may include a fluoroscopic imaging system, which is merely used to confirm placement of a medical tool near a target prior to biopsy or treatment of a target.

The computer system 122 may be any suitable computer system including a processor and storage medium, wherein the processor is capable of executing instructions stored on the storage medium. The computer system 122 may further include a database configured to store patient data, preoperative CT data sets, e.g., preoperative CT images captured according to functional respiratory imaging (FRI), navigation plans, optionally fluoroscopic data sets including fluoroscopic images and video, optionally fluoroscopic 3D reconstruction, and any other such data. Although not explicitly illustrated, the computer system 122 may include inputs for, or may otherwise be configured to receive, preoperative CT data sets, optional fluoroscopic images/video, and other data described herein. Additionally, the computer system 122 includes a display configured to display graphical user interfaces. The computer system 122 may be connected to one or more networks through which one or more databases may be accessed.

With respect to the planning phase, the computer system 122 utilizes previously acquired CT image data for determining regular motion of a patient, e.g., motion caused by respiration, utilizes the same or different previously acquired CT image data for generating and viewing a three-dimensional model or rendering of patient P's airways, enables the identification of a target on the three-dimensional model (automatically, semi-automatically, or manually), and allows for determining a pathway through patient P's airways to tissue located at and around the target. More specifically, the CT images acquired from the previous CT scans are processed and assembled into a three-dimensional CT volume, which is then utilized to generate a three-dimensional model of patient P's airways. The three-dimensional model may be displayed on a display associated with the computer system 122, or in any other suitable fashion. Using the computer system 122, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient P's airways to access tissue located at the target can be made. Once selected, the pathway plan, three-dimensional model, and images derived therefrom, can be saved and exported to a navigation system for use during the navigation phase(s). One such planning software is the ILLUMISITE® planning suite currently sold by Medtronic PLC.

With respect to the navigation phase, a six degrees-of-freedom electromagnetic locating or a tracking system 114, or other suitable system for determining location, is utilized for performing registration of the images and the pathway for navigation, although other configurations are also contemplated. The tracking system 114 includes the tracking module 116, the patient motion sensors 118, which may also be used as patient motion sensors, and the transmitter mat 120 (including the markers). The tracking system 114 is configured for use with a locatable guide 110 and particularly EM sensor 104. As described above, the locatable guide 110 and the EM sensor 104 are configured for insertion through the catheter 102 into patient P's airways (either with or without the bronchoscope 108) and are selectively lockable relative to one another via a locking mechanism.

The transmitter mat 120 is positioned beneath patient P. The transmitter mat 120 generates an electromagnetic field around at least a portion of the patient P within which the positions of the patient motion sensors 118 and the EM sensor 104 can be determined with use of a tracking module 116. A second EM sensor 126 may also be incorporated into the end of the catheter 102. The second EM sensor 126 may be a five degree-of-freedom sensor or a six degree-of-freedom sensor. One or more of the patient motion sensors 118 are attached to the chest of the patient P or at suitable positions on the patient's body that optimize the sensing of the motion of the patient. The six degrees of freedom coordinates of the patient motion sensors 118 are sent to the computer system 122 (which includes the appropriate software) where they are used to calculate a patient coordinate frame of reference. The six degrees of freedom coordinates of the patient motion sensors 118 are also sent to the computer system 122 where they are used to track the real-time motion of the patient, which may be caused by respiration cycles, e.g., inspiration and expiration, of the patient. Registration is generally performed to coordinate locations of the three-dimensional model and two-dimensional images from the planning phase, with the patient P's airways as observed through the bronchoscope 108, and allow for the navigation phase to be undertaken with precise knowledge of the location of the EM sensor 104, even in portions of the airway where the bronchoscope 108 cannot reach.

Registration of the patient P's location on the transmitter mat 120 may be performed by moving the EM sensor 104 through the airways of the patient P. More specifically, data pertaining to locations of the EM sensor 104, while locatable guide 110 is moving through the airways, is recorded using the transmitter mat 120, the patient motion sensors 118, and the tracking system 114. A shape resulting from this location data is compared to an interior geometry of passages of the three-dimensional model generated in the planning phase, and a location correlation between the shape and the three-dimensional model based on the comparison is determined, e.g., utilizing the software on the computer system 122. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that the locatable guide 110 remains located in non-tissue space in patient P's airways. Alternatively, a manual registration technique may be employed by navigating the bronchoscope 108 with the EM sensor 104 to pre-specified locations in the lungs of the patient P, and manually correlating the images from the bronchoscope to the model data of the three-dimensional model.

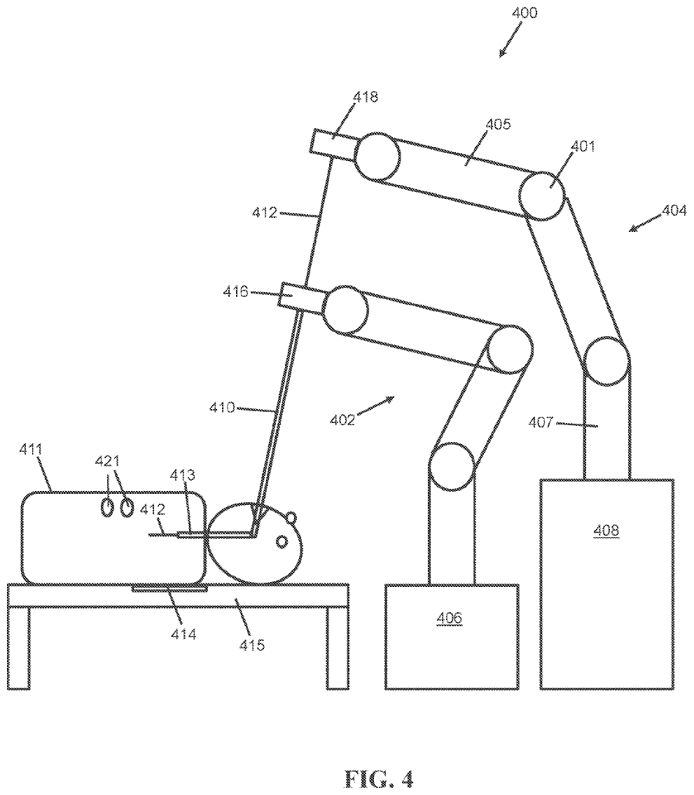

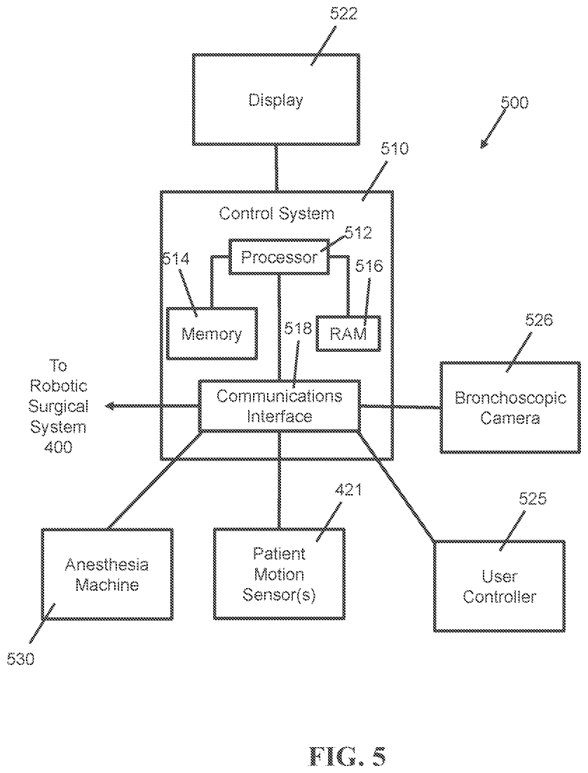

Though described herein with respect to EMN systems using EM sensors, the instant disclosure is not so limited and may be used in conjunction with flexible sensors, ultrasonic sensors, or other suitable motion sensors. Additionally, the methods described herein may be used in conjunction with robotic systems such that robotic actuators drive the catheter 102, bronchoscope 108, or other medical tool proximate the target. An example of a robotic system is illustrated in FIGS. 4 and 5.

Following registration of the patient P to the image data and pathway plan, a user interface is displayed in the navigation software which sets for the pathway that the clinician is to follow to reach the target. Once the catheter 102 has been successfully navigated proximate the target as depicted on the user interface, the locatable guide 110 may be unlocked from the catheter 102 and removed, leaving the catheter 102 in place as a guide channel for guiding medical tools including without limitation, optical systems, ultrasound probes, marker placement tools, biopsy tools, ablation tools (i.e., microwave ablation tools), laser probes, cryogenic probes, sensor probes, and aspirating needles to the target. A medical tool may be then inserted through the catheter 102 and navigated to the target or to a specific area adjacent to the target.

Prior to inserting the medical tool through the catheter 102, a local registration process may optionally be performed for each target to reduce the CT-to-body divergence. In a capture phase of the local registration process, a sequence of fluoroscopic images may be captured and acquired via the imaging system 124, optionally by a user and according to directions displayed via the computer system 122. A fluoroscopic 3D reconstruction may be then generated via the computer system 122. The generation of the fluoroscopic 3D reconstruction is based on the sequence of fluoroscopic images and the projections of structure of markers incorporated with transmitter mat 120 on the sequence of fluoroscopic images. One or more slices of the 3D reconstruction may be then generated based on the pre-operative CT scan and via the computer system 122. The one or more slices of the 3D reconstruction and the fluoroscopic 3D reconstruction may be then displayed to the user on a display via the computer system 122, optionally simultaneously. The slices of 3D reconstruction may be presented on the user interface in a scrollable format where the user is able to scroll through the slices in series.

In a marking phase of the local registration process, the clinician may be directed to identify and mark the target while using the slices of the 3D reconstruction as a reference. The user may also be directed to identify and mark the navigation catheter tip in the sequence of fluoroscopic 2D images. An offset between the location of the target and the navigation catheter tip may be then determined or calculated via the computer system 122. The offset may be then utilized, via the computer system 122, to correct the location and/or orientation of the navigation catheter on the display with respect to the target and/or correct the registration between the three-dimensional model and tracking system 114 in the area of the target and/or generate a local registration between the three-dimensional model and the fluoroscopic 3D reconstruction in the target area.

In an optional confirmation phase of the local registration process, a fluoroscopic 3D reconstruction is displayed in a confirmation screen. The confirmation screen may include a slider that may be selected and moved by the user to review a video loop of the fluoroscopic 3D reconstruction, which shows the marked target and navigation catheter tip from different perspectives. After confirming that there are marks on the target and navigation catheter tip throughout the video, the clinician may select an “Accept” button, at which point the local registration process ends and the position of the navigation catheter is updated. The clinician may then use the navigation views in, for example, the peripheral navigation screen to fine tune the alignment of the navigation catheter to the target before beginning an endoscopic procedure.

After the local registration process, the clinician or robotic arm may insert a medical tool in the catheter 102 and advance the medical tool towards the target. While advancing the medical tool towards the target, the clinician may view a user interface screen which includes a 3D medical tool tip view of a 3D model of a target, in which the 3D model of the target is moved according to motion of the target determined from pre-operative CT scans of a patient's motion and the motion sensed by the patient motion sensors. This user interface screen allows the clinician to not only see the medical tool in real-time, but also allows the clinician to see whether the medical tool is aligned with the moving target. The user interface screen may also provide a graphical indication of whether the medical tool is aligned in three-dimensions with the target. For example, when the medical tool is aligned in three-dimensions with the target, the user interface shows the target overlay in a first color, e.g., green. On the other hand, when the medical tool is not aligned with the target in three dimensions, the user interface shows the target overlay in a second color different from the first color, e.g., orange or red.

FIG. 2 is a flowchart of an example of a method for visualizing a medical tool, e.g., a catheter, relative to a moving target during respiration cycles of a patient. At block 202, preoperative 3D images of motion of the patient are received. The preoperative 3D images may be computed tomography (CT) images, cone beam computed tomography (CBCT) images, or magnetic resonance imaging (MRI) images. These images may be captured according to functional respiratory imaging (FRI). FRI may include acquiring low dose, high-resolution CT scans of a patient's lungs, segmenting the CT scans to obtain 3D geometries, and performing functional simulations, e.g., quantifying airflow through the lungs using computational fluid dynamics (CFD), which provides, among other things, detailed information regarding the motion of all portions of the lungs during one or more respiratory cycles. At block 204, navigation of a medical tool towards a target is tracked using an EM sensor disposed on the medical tool, e.g., disposed on the tip of the medical tool as described herein.

At block 206, motion of the patient is intraoperatively tracked in real-time using patient motion information. The patient motion information may be received from one or more motion sensors disposed on and/or in the patient's chest, from an anesthesia machine, or from any suitable system for tracking the patient's motion or respiratory cycle. The one or more motion sensors may be EM sensors, e.g., the patent motion sensors 118 of the system 100 of FIG. 1, tracked in real-time by the tracking system 114 shown in FIG. 1. At block 208, 3D motion of target is determined based on preoperative 3D images and tracked patient motion. Determining the 3D motion of the target in the patient may include registering the preoperative 3D images with the tracked patient motion.

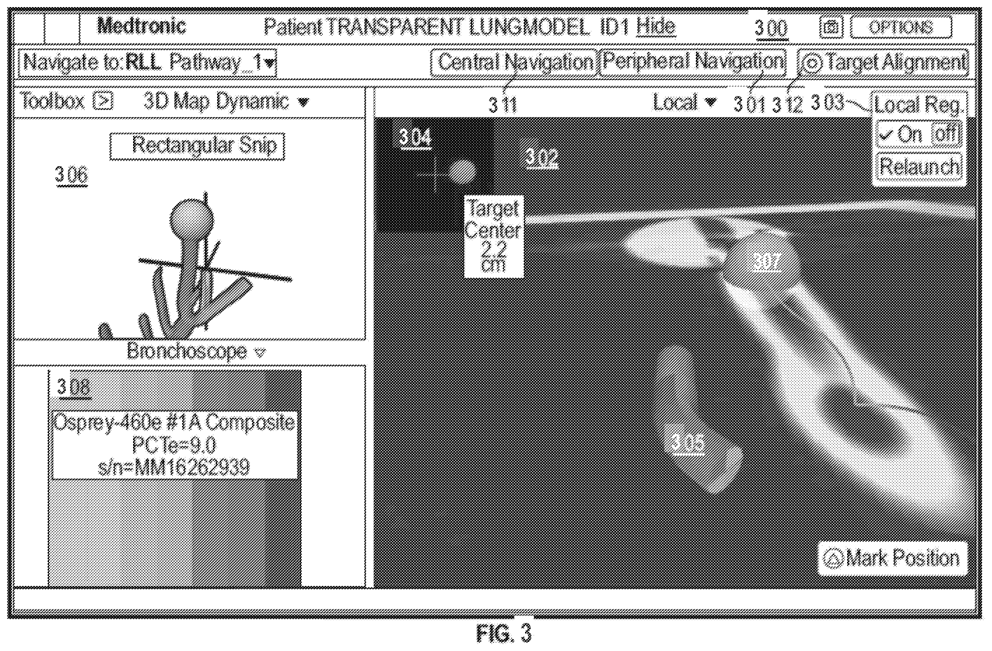

After the 3D motion of the target is determined at block 208, the tip of the medical tool is displayed relative to a moving target during motion of the patient using the EM sensor disposed on the medical tool and the 3D motion of the target at block 210. In aspects, the method 200 may include displaying, in a user interface, an indicator of at least one direction in which to navigate the medical tool to reach the target. In one example, the tip of the medical tool and the target may be displayed in the user interface 300 of FIG. 3.

FIG. 3 shows a peripheral navigation screen 301 associated with the “Peripheral Navigation” tab of the user interface 300 of FIG. 3. The peripheral navigation screen 301 includes a local CT view 302, a 3D navigation catheter tip view 304, a 3D map view 306, and a bronchoscope view 308. The peripheral navigation screen 301 also includes local registration user controls 303 enabling the user to apply local registration and/or relaunch local registration. The user interface 300 also includes a “Central Navigation” tab 311 and a “Target Alignment” tab 312, which may be individually selected to perform central navigation or target alignment, respectively. The tip portion 305 of a medical tool is displayed relative to a moving target 307. In aspects, the tip portion 305 may be displayed as being stationary, while the target 307 may be moved according to the determined 3D motion of the target.

FIG. 4 is a block diagram that illustrates a robotic surgical system 400 in accordance with aspects of this disclosure. The robotic surgical system 400 includes a first robotic arm 402 and a second robotic arm 404 attached to robotic arm bases 406 and 408, respectively. The first robotic arm 402 and the second robotic arm 404 include a first end effector 416 and a second end effector 418, respectively. The end effectors 416, 418 may include robotic manipulators or grippers suitable for operating the endoscopic catheters and medical tools of this disclosure. The first end effector 416 operates one or more tools 412, including a biopsy tool and/or a flexible endoscope or bronchoscope. The second end effector 418 operates a sheath or catheter 410, which may include one or more channels for receiving and guiding the one or more tools 412. The robotic surgical system 400 may further include an electromagnetic (EM) generator 414, which is configured to generate an EM field, which is sensed by an EM sensor incorporated into or disposed on the medical tool and the EM patient motion sensors 421 are disposed on and/or in the patient. In aspects, the EM generator 414 may be embedded in the operating table 415 or may be incorporated into a pad that may be placed between the operating table 415 and the patient 411.

The first and second robotic arms 402, 404 may be controlled to align the end effectors 416 and 418 such that proximal end portion of the catheter 410 is distal to the proximal end portions of the one or more tools 412, and such that the one or more tools 412 remain axially aligned with catheter 410.

In one aspect, the first robotic arm 402 inserts the catheter 410 through, for example, a tracheal tube (not shown) in the mouth of the patient 411, and into the bronchial system of the patient 411. Then, the second robotic arm 404 inserts the one or more tools 412, e.g., a biopsy tool, through the catheter 102 to reach a target within the bronchial system of the patient 411. The first and second robotic arms 402, 404 may move the catheter 410 and one or more tools 412, e.g., a biopsy tool, axially relative to each other and into or out of the patient 411 under the control of a surgeon (not shown) at a control console (not shown).

A navigation phase may include advancing catheter 410 along with the one or more tools 412 into the patient 411, and then advancing the one or more tools 412 beyond the distal end of the catheter 410 to reach a desired destination such as a target. Other modes of navigation may be used, such as by using a guide wire through a working channel of the catheter 410. The surgeon may use a visual guidance modality or a combination of visual guidance modalities to aid in navigation and performing the biopsy procedure, such as fluoroscopy, video, computed tomography (CT), or magnetic resonance imaging (MRI). In aspects, the one or more tools 412 are deployed through longitudinally-aligned working channels within the catheter 410 to perform the biopsy procedure or any other desired procedures. In aspects, the robotic arms 402, 404 may include three joints 401 and three-arm segments 405. In other aspect, the robotic arms 402, 404 may include greater than or less than three joints 401 and three arm segments 405.

FIG. 5 is a block diagram that illustrates a robotic control system 500 for controlling the robotic surgical system 400 of FIG. 4. The robotic control system 500 includes a control system 510, which controls the robotic surgical system 400. For example, the control system 510 may execute the method 600 of FIG. 6 described herein. The control system 510 may interface with a display 522, a user controller 525, and an endoscopic or bronchoscopic camera 526. The control system 510 may be coupled to the robotic surgical system 400, directly or indirectly, e.g., by wireless communication. The control system 510 includes a processor 512, a memory 514 coupled to the processor 512, a random access memory (RAM) 516 coupled to the processor 512, and a communications interface 518 coupled to the processor 512. The processor 512 may include one or more hardware processors. The control system 510 may be a stationary computer, such as a personal computer, or a portable computer such as a tablet computer. Alternatively, the control system 510 may be incorporated into one of the robotic arm bases 406, 408. The control system 510 may also interface with a user controller 525, which may be used by a surgeon to control the robotic arm system 524 to perform a biopsy procedure.

It should be appreciated by those skilled in the art that the memory 514 may be any computer-readable storage media that can be accessed by the processor 512. That is, computer readable storage media may include non-transitory, volatile, and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray, or other optical storage, magnetic cassettes, magnetic tape, magnetic disk storage or other magnetic storage devices, or any other medium which may be used to store the desired information, and which may be accessed by processor 512.

An application stored in the memory 514 may, when executed by processor 512, cause display 522 to present a user interface (not shown). The user interface may be configured to present to the user bronchoscopic images from the bronchoscopic camera 526. Optionally, the user interface may be further configured to direct the user to select the target by, among other things, identifying and marking the target in the image data.

Communications interface 518 may be configured to connect to a network such as a local area network (LAN) consisting of a wired network and/or a wireless network, a wide area network (WAN), a wireless mobile network, a Bluetooth network, and/or the internet. Communications interface 518 may be used to connect between the control system 510 and the bronchoscopic camera 526. Communications interface 518 may be also used to receive image data from the memory 514 and path planning data. Communications interface 518 may also be coupled to or in communication with one or more patient motion sensors 421 and/or an anesthesia machine 530 to receive patient motion information such as a patient's respiratory cycle. The control system 510 may also include an input device (not shown), which may be any device through which a user may interact with the control system 510, such as, for example, a mouse, keyboard, foot pedal, touch screen, and/or voice interface. The control system 510 may also include an output module (not shown), which may include any connectivity port or bus, such as, for example, parallel ports, serial ports, universal serial busses (USB), or any other similar connectivity port known to those skilled in the art.

FIG. 6 is a flowchart of an example of a method for controlling one or more robotic arms, e.g., robotic arms 402, 404 of FIG. 4, to orient and advance a catheter relative to a moving target during respiration of a patient. At block 602, preoperative 3D images of motion of the patient are received, for example, by the computer system 122. The preoperative 3D images may be captured by a 3D imaging system, e.g., CT imaging system, during one or more respiratory cycles of the patient. At block 604, navigation of a medical tool towards a target is tracked using a first EM sensor, which may be disposed on the tip of the medical tool. The medical tool may be an extended working channel or a biopsy tool. At block 606, the motion of patient is intraoperatively tracked using patient motion information. The patient motion information may be received from one or more second EM sensors disposed on and/or in the patient, from an anesthesia machine, or from any suitable system for tracking the patient's motion or respiratory cycle. In aspects, the one or more second EM sensors are disposed on and/or in the patient's chest and are configured to track the motion of the patient's chest during one or more respiratory cycles. At block 608, 3D motion of target is determined based on the preoperative 3D images and the tracked patient motion. The 3D motion of the target may be represented by a functional model and may be determined by registering the preoperative 3D images to the 3D motion of the target. In aspects, the method 600 may include segmenting the target from the sequence of preoperative 3D images.

After the 3D motion of the target is determined at block 608, the orientation of tip of catheter is controlled, e.g., via a robotic arm, to align with or track the target during motion of patient using the first EM sensor and 3D motion of the target at block 610. In aspects, the computer system 122 may determine whether the medical tool, e.g., an extended working channel or a biopsy catheter, is not aligned with the target. If the computer system 122 determines that the medical tool is not aligned with the target, the computer system 122 may generate an alarm. In aspects, the computer system 122 may control the robotic arm to navigate the medical tool through a luminal network of the patient.

Reference is now made to FIG. 7, which is a schematic diagram of a system 700 configured for use with the methods of the disclosure including the methods of FIGS. 2 and 6. The system 700 may include a workstation 701, and optionally an imaging system 715, e.g., a fluoroscopic imaging system and/or a CT imaging system for capturing preoperative 3D images. In some aspects, the workstation 701 may be coupled with the imaging system 715, directly or indirectly, e.g., by wireless communication. The workstation 701 may include a memory 702, a processor 704, a display 706 and an input device 710. The processor 704 may include one or more hardware processors. The workstation 701 may optionally include an output module 712 and a network interface 1008. The memory 702 may store an application 718 and image data 714. The application 718 may include instructions executable by the processor 704 for executing the methods of the disclosure including the methods of FIGS. 2 and 6.

The application 718 may further include a user interface 716. The image data 714 may include preoperative CT image data, fluoroscopic image data, or fluoroscopic 3D reconstruction data. The processor 704 may be coupled with the memory 702, the display 706, the input device 710, the output module 712, the network interface 708, and the imaging system 715. The workstation 701 may be a stationary computer system, such as a personal computer, or a portable computer system such as a tablet computer. The workstation 701 may embed multiple computers.

The memory 702 may include any non-transitory computer-readable storage media for storing data and/or software including instructions that are executable by the processor 704 and which control the operation of the workstation 701 and, in some aspects, may also control the operation of the imaging system 715. The imaging system 715 may be used to capture a sequence of preoperative CT images of a portion of a patient's body, e.g., the lungs, as the portion of the patient's body moves, e.g., as the lungs move during a respiratory cycle. Optionally, the imaging system 715 may include a fluoroscopic imaging system that captures a sequence of fluoroscopic images based on which a fluoroscopic 3D reconstruction is generated and to capture a live 2D fluoroscopic view to confirm placement of a medical tool. In one aspect, the memory 702 may include one or more storage devices such as solid-state storage devices, e.g., flash memory chips. Alternatively, or in addition to the one or more solid-state storage devices, the memory 702 may include one or more mass storage devices connected to the processor 704 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 704. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 701.

The application 718 may, when executed by the processor 704, cause the display 706 to present the user interface 716. The user interface 716 may be configured to present to the user a single screen including a three-dimensional (3D) view of a 3D model of a target from the perspective of a tip of a medical tool, a live two-dimensional (2D) fluoroscopic view showing the medical tool, and a target mark, which corresponds to the 3D model of the target, overlaid on the live 2D fluoroscopic view. The user interface 716 may be further configured to display the target mark in different colors depending on whether the medical tool tip is aligned with the target in three dimensions.

The network interface 708 may be configured to connect to a network such as a local area network (LAN) consisting of a wired network and/or a wireless network, a wide area network (WAN), a wireless mobile network, a Bluetooth network, and/or the Internet. The network interface 708 may be used to connect between the workstation 701 and the imaging system 715. The network interface 708 may be also used to receive the image data 714. The input device 710 may be any device by which a user may interact with the workstation 701, such as, for example, a mouse, keyboard, foot pedal, touch screen, and/or voice interface. The output module 712 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

While detailed aspects are disclosed herein, the disclosed aspects are merely examples of the disclosure, which may be embodied in various forms and aspects. For example, aspects of visualization and robotic systems, which incorporate an electromagnetic navigation system are disclosed herein; however, the visualization and robotic systems and methods may be applied to other navigation or tracking systems or methods known to those skilled in the art. Therefore, specific structural and functional details disclosed herein are not to be interpreted as limiting, but merely as a basis for the claims and as a representative basis for teaching one skilled in the art to variously employ the disclosure in virtually any appropriately detailed structure.

While aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of aspects. Those skilled in the art will envision other modifications within the scope and spirit of the claims appended hereto.

What is claimed is:

1. A method comprising:
receiving preoperative three dimensional (3D) images of motion of a patient including a target;
navigating a medical tool near the target based on information from a position sensor disposed on the medical tool;
receiving patient motion information;
tracking intraoperative 3D motion of the patient based on the patient motion information, yielding tracked patient motion;
determining 3D motion of the target in the patient based on the preoperative 3D images and the tracked patient motion; and
controlling the medical tool to track the 3D motion of the target.

2. The method of claim 1, wherein the preoperative 3D images are computed tomography (CT) images, cone beam computed tomography (CBCT) images, or magnetic resonance imaging (MRI) images.

3. The method of claim 1, wherein the patient motion information is received from electromagnetic (EM) motion sensors or an anesthesia machine.

4. The method of claim 1, further comprising wherein the preoperative 3D images are captured using functional respiratory imaging (FRI).

5. The method of claim 1, wherein determining the 3D motion of the target in the patient includes registering the preoperative 3D images with the tracked patient motion.

6. An endoluminal navigation method comprising:
receiving preoperative 3D images of motion of a patient including a target;
displaying guidance for navigating a medical tool near the target based on the preoperative 3D images and information from a position sensor disposed on the medical tool;
receiving patient motion information;
tracking motion of the patient based on the patient motion information, yielding tracked patient motion;
determining 3D motion of the target in the patient based on the preoperative 3D images and the tracked patient motion; and
displaying the 3D motion of the target relative to a tip of the medical tool.

7. The endoluminal navigation method of claim 6, further comprising capturing the preoperative 3D images during a respiratory cycle of the patient.

8. The endoluminal navigation method of claim 6, further comprising displaying an indicator of at least one direction in which to navigate the medical tool to reach the target.

9. The endoluminal navigation method of claim 6, further comprising:
segmenting the target from the preoperative 3D images, yielding segmented targets; and
determining positions of the target in a reference frame of the preoperative 3D images based on the segmented targets.

10. The endoluminal navigation method of claim 6, further comprising registering the preoperative 3D images to tracked patient motion.

11. A robotic endoluminal navigation system comprising:
a robotic arm configured to hold and navigate a medical tool;
an electromagnetic (EM) field generator configured to generate an electromagnetic field;
a first EM sensor disposed at a tip of the medical tool;
one or more second EM sensors disposed on a patient;
a processor; and
a memory having stored thereon instructions, which, when executed by the processor, cause the processor to:
receive preoperative 3D images of motion of the patient;
track navigation of the medical tool towards a target using the first EM sensor;
intraoperatively track motion of the patient using the one or more second EM sensors disposed on the patient, yielding tracked patient motion;
determine 3D motion of the target in the patient based on the preoperative 3D images and the tracked patient motion; and
control the medical tool to align with the target during motion of the patient using the first EM sensor and the 3D motion of the target.

12. The robotic endoluminal navigation system of claim 11, wherein the preoperative 3D images are captured during at least one respiratory cycle of the patient.

13. The robotic endoluminal navigation system of claim 11, wherein the instructions, when executed by the processor, further cause the processor to control the robotic arm to navigate the medical tool towards the target during patient motion.

14. The robotic endoluminal navigation system of claim 11, wherein the one or more second EM sensors are disposed on a chest of the patient and configured to track the motion of the chest of the patient during at least one respiratory cycle.

15. The robotic endoluminal navigation system of claim 11, wherein the instructions, when executed by the processor, further cause the processor to control the robotic arm to navigate the medical tool through a luminal network of the patient.

16. The robotic endoluminal navigation system of claim 11, wherein the medical tool is an extended working channel or a biopsy tool.

17. An endoluminal navigation system comprising:
an electromagnetic (EM) field generator configured to generate an electromagnetic field;
a first EM sensor disposed at a tip of a medical tool;
one or more second EM sensors disposed on a chest of a patient;
a display;
a processor; and
a memory having stored thereon instructions, which, when executed by the processor, cause the processor to:
receive preoperative 3D images of motion of the patient;
track navigation of the medical tool towards a target using the first EM sensor;
intraoperatively track motion of the patient using the one or more second EM sensors disposed on the chest of the patient, yielding tracked patient motion;
determine 3D motion of the target in the patient based on the preoperative 3D images and the tracked patient motion; and
display on the display the target and the tip of the medical tool relative to the target during motion of the patient using the first EM sensor and the 3D motion of the target.

18. The endoluminal navigation system of claim 17, wherein the instructions, when executed by the processor, further cause the processor to display an indicator of at least one direction in which to navigate the medical tool to reach the target.

19. The endoluminal navigation system of claim 17, wherein the instructions, when executed by the processor, further cause the processor to segment the target from the preoperative 3D images.

20. The endoluminal navigation system of claim 17, wherein the instructions, when executed by the processor, further cause the processor to register the preoperative 3D images to the 3D motion of the target.
